SEQUENCE LISTING (192 sequences)

SEQ ID NO: 1 - DNA, 39 bp, Artificial Sequence
  PCR primer
tgtaaaacgacggccagtttgtccataatctctggtgcc

SEQ ID NO: 2 - DNA, 20 bp, Artificial Sequence
  PCR primer
tcacttggccacatgtctct

SEQ ID NO: 3 - DNA, 39 bp, Artificial Sequence
  PCR primer
tgtaaaacgacggccagtgggtaggatcccaactagagc

SEQ ID NO: 4 - DNA, 20 bp, Artificial Sequence
  PCR primer
accaaaccttagaggccacc

SEQ ID NO: 5 - DNA, 38 bp, Artificial Sequence
  PCR primer
tgtaaaacgacggccagtgcaaatgcaaatcctccaat

SEQ ID NO: 6 - DNA, 20 bp, Artificial Sequence
  PCR primer
atcccagttctgcacaatcc

SEQ ID NO: 7 - DNA, 38 bp, Artificial Sequence
  PCR primer
tgtaaaacgacggccagtagcgaccagaaatacctcca

SEQ ID NO: 8 - DNA, 20 bp, Artificial Sequence
  PCR primer
gctgcctcagagtctccttc

SEQ ID NO: 9 - DNA, 38 bp, Artificial Sequence
  PCR primer
tgtaaaacgacggccagtgaggatgttgcttgtgatgc

SEQ ID NO: 10 - DNA, 20 bp, Artificial Sequence
  PCR primer
tttctggaaatgttgccctt

SEQ ID NO: 11 - DNA, 38 bp, Artificial Sequence
  PCR primer
tgtaaaacgacggccagtacctccctggtaacccagac

SEQ ID NO: 12 - DNA, 20 bp, Artificial Sequence
  PCR primer
ttgaaaccctttgttgcaga

SEQ ID NO: 13 - DNA, 38 bp, Artificial Sequence
  PCR primer
tgtaaaacgacggccagtcgacaaagaaacgggaagag

SEQ ID NO: 14 - DNA, 20 bp, Artificial Sequence
  PCR primer
cgacaagggctggatttaga

SEQ ID NO: 15 - DNA, 38 bp, Artificial Sequence
  PCR primer
tgtaaaacgacggccagtcgaggagggacaacattcat

SEQ ID NO: 16 - DNA, 20 bp, Artificial Sequence
  PCR primer
cagcatgggagctacaaaca

SEQ ID NO: 17 - DNA, 38 bp, Artificial Sequence
  PCR primer
tgtaaaacgacggccagtatgggttgcagaagaggatg

SEQ ID NO: 18 - DNA, 21 bp, Artificial Sequence
  PCR primer
ttgccatatactcccatgtcc

SEQ ID NO: 19 - DNA, 38 bp, Artificial Sequence
  PCR primer
tgtaaaacgacggccagtagcagcaacaacattaggca

SEQ ID NO: 20 - DNA, 20 bp, Artificial Sequence
  PCR primer
gaattgcatctgaaggaggg

SEQ ID NO: 21 - DNA, 38 bp, Artificial Sequence
  PCR primer
tgtaaaacgacggccagttcatcataacggaaggtggg

SEQ ID NO: 22 - DNA, 21 bp, Artificial Sequence
  PCR primer
agctgccatgtcataagctgt

SEQ ID NO: 23 - DNA, 38 bp, Artificial Sequence
  PCR primer
tgtaaaacgacggccagtcgctagggattgtgatcgtt

SEQ ID NO: 24 - DNA, 20 bp, Artificial Sequence
  PCR primer
gttgttgttaccgcctccac

SEQ ID NO: 25 - DNA, 38 bp, Artificial Sequence
  PCR primer
tgtaaaacgacggccagttcaggcattcccttcaaagt

SEQ ID NO: 26 - DNA, 20 bp, Artificial Sequence
  PCR primer
cgtgaaagtgaagcgaccta

SEQ ID NO: 27 - DNA, 38 bp, Artificial Sequence
  PCR primer
tgtaaaacgacggccagtggtggaaggaagtgcaagaa

SEQ ID NO: 28 - DNA, 20 bp, Artificial Sequence
  PCR primer
gcccaaataaaccatgagga

SEQ ID NO: 29 - DNA, 38 bp, Artificial Sequence
  PCR primer
tgtaaaacgacggccagtatccatgccagattctccac

SEQ ID NO: 30 - DNA, 20 bp, Artificial Sequence
  PCR primer
agccatttctacgcttgcag

SEQ ID NO: 31 - DNA, 38 bp, Artificial Sequence
  PCR primer
tgtaaaacgacggccagttccacgaccttcaacaacaa

SEQ ID NO: 32 - DNA, 20 bp, Artificial Sequence
  PCR primer
ggcagaagagatgatagccg

SEQ ID NO: 33 - DNA, 38 bp, Artificial Sequence
  PCR primer
tgtaaaacgacggccagttgccgagtgctgattctatg

SEQ ID NO: 34 - DNA, 20 bp, Artificial Sequence
  PCR primer
gaatttgcattccttggtgc

SEQ ID NO: 35 - DNA, 38 bp, Artificial Sequence
  PCR primer
tgtaaaacgacggccagtgctggacttgagaggtgtgg

SEQ ID NO: 36 - DNA, 20 bp, Artificial Sequence
  PCR primer
tgatgaccacctgttgccta

SEQ ID NO: 37 - DNA, 38 bp, Artificial Sequence
  PCR primer
tgtaaaacgacggccagttgagaagctccatcaagggt

SEQ ID NO: 38 - DNA, 22 bp, Artificial Sequence
  PCR primer
cgattcaaatggtcctttcttc

SEQ ID NO: 39 - DNA, 39 bp, Artificial Sequence
  PCR primer
tgtaaaacgacggccagtaggtgaaggttcttgaggagg

SEQ ID NO: 40 - DNA, 20 bp, Artificial Sequence
  PCR primer
cgtcaaagggatcaccagat

SEQ ID NO: 41 - DNA, 38 bp, Artificial Sequence
  PCR primer
tgtaaaacgacggccagtgttccgggtacaaagcatgt

SEQ ID NO: 42 - DNA, 20 bp, Artificial Sequence
  PCR primer
ccaaggtgagacactcggtc

SEQ ID NO: 43 - DNA, 38 bp, Artificial Sequence
  PCR primer
tgtaaaacgacggccagtaacagagactgcaacagcca

SEQ ID NO: 44 - DNA, 20 bp, Artificial Sequence
  PCR primer
agcgtaagttccaagccaga

SEQ ID NO: 45 - DNA, 38 bp, Artificial Sequence
  PCR primer
tgtaaaacgacggccagttatcgacccaaatgcaaaca

SEQ ID NO: 46 - DNA, 20 bp, Artificial Sequence
  PCR primer
acagcctttacgcatccaaa

SEQ ID NO: 47 - DNA, 38 bp, Artificial Sequence
  PCR primer
tgtaaaacgacggccagttctaaggcagtccttgtggg

SEQ ID NO: 48 - DNA, 20 bp, Artificial Sequence
  PCR primer
ttgagttgccatcaggttca

SEQ ID NO: 49 - DNA, 38 bp, Artificial Sequence
  PCR primer
tgtaaaacgacggccagttgggatcagacagtccacaa

SEQ ID NO: 50 - DNA, 20 bp, Artificial Sequence
  PCR primer
ggaacagagccagaacggta

SEQ ID NO: 51 - DNA, 38 bp, Artificial Sequence
  PCR primer
tgtaaaacgacggccagtggccatcatcacaaggagtt

SEQ ID NO: 52 - DNA, 20 bp, Artificial Sequence
  PCR primer
tcatgcctttgcatcttcag

SEQ ID NO: 53 - DNA, 38 bp, Artificial Sequence
  PCR primer
tgtaaaacgacggccagtcatgccaggatccattaacc

SEQ ID NO: 54 - DNA, 20 bp, Artificial Sequence
  PCR primer
cactgagtcctcctcctgct

SEQ ID NO: 55 - DNA, 38 bp, Artificial Sequence
  PCR primer
tgtaaaacgacggccagtaaacgttggaacaagttggg

SEQ ID NO: 56 - DNA, 20 bp, Artificial Sequence
  PCR primer
agcatttgtttggaagtggg

SEQ ID NO: 57 - DNA, 39 bp, Artificial Sequence
  PCR primer
tgtaaaacgacggccagtcgtagggatggagacaatgag

SEQ ID NO: 58 - DNA, 20 bp, Artificial Sequence
  PCR primer
aatgtagctggtggtggcat

SEQ ID NO: 59 - DNA, 38 bp, Artificial Sequence
  PCR primer
tgtaaaacgacggccagtttgtgtgcgttggtcaagat

SEQ ID NO: 60 - DNA, 20 bp, Artificial Sequence
  PCR primer
acgcttgagttcggcaatag

SEQ ID NO: 61 - DNA, 38 bp, Artificial Sequence
  PCR primer
tgtaaaacgacggccagttcgggagctggagtaagaaa

SEQ ID NO: 62 - DNA, 20 bp, Artificial Sequence
  PCR primer
ggtaattcaggatcgggtca

SEQ ID NO: 63 - DNA, 38 bp, Artificial Sequence
  PCR primer
tgtaaaacgacggccagttgctgtcaaaggtgattgga

SEQ ID NO: 64 - DNA, 20 bp, Artificial Sequence
  PCR primer
atcgaggaaagacgacgatg

SEQ ID NO: 65 - DNA, 38 bp, Artificial Sequence
  PCR primer
tgtaaaacgacggccagtgagtctaacacagccgcaca

SEQ ID NO: 66 - DNA, 20 bp, Artificial Sequence
  PCR primer
cccttcacttcctgattcca

SEQ ID NO: 67 - DNA, 38 bp, Artificial Sequence
  PCR primer
tgtaaaacgacggccagttccgacaacaattcgaacac

SEQ ID NO: 68 - DNA, 20 bp, Artificial Sequence
  PCR primer
gtcctcaatggcaacatcct

SEQ ID NO: 69 - DNA, 38 bp, Artificial Sequence
  PCR primer
tgtaaaacgacggccagtccagtgaacaagcctggaat

SEQ ID NO: 70 - DNA, 20 bp, Artificial Sequence
  PCR primer
caaatcggaagctcagaagg

SEQ ID NO: 71 - DNA, 38 bp, Artificial Sequence
  PCR primer
tgtaaaacgacggccagttcatgcaaacttctgctgct

SEQ ID NO: 72 - DNA, 20 bp, Artificial Sequence
  PCR primer
ccactgtgatggctgaggta

SEQ ID NO: 73 - DNA, 38 bp, Artificial Sequence
  PCR primer
tgtaaaacgacggccagtattcttgatgcacctcccac

SEQ ID NO: 74 - DNA, 20 bp, Artificial Sequence
  PCR primer
gccatatttgagttcccagc

SEQ ID NO: 75 - DNA, 38 bp, Artificial Sequence
  PCR primer
tgtaaaacgacggccagtacaaccaccaatgatgacga

SEQ ID NO: 76 - DNA, 20 bp, Artificial Sequence
  PCR primer
atgcaacttcccataccagc

SEQ ID NO: 77 - DNA, 38 bp, Artificial Sequence
  PCR primer
tgtaaaacgacggccagttgaaattgaaaggccaccat

SEQ ID NO: 78 - DNA, 20 bp, Artificial Sequence
  PCR primer
ttcaccgggaagaagtgaac

SEQ ID NO: 79 - DNA, 39 bp, Artificial Sequence
  PCR primer
tgtaaaacgacggccagtttggatctcctctgatcctga

SEQ ID NO: 80 - DNA, 20 bp, Artificial Sequence
  PCR primer
cttacctttcttcccgtccc

SEQ ID NO: 81 - DNA, 38 bp, Artificial Sequence
  PCR primer
tgtaaaacgacggccagttctttgtcaccagacgcaac

SEQ ID NO: 82 - DNA, 20 bp, Artificial Sequence
  PCR primer
gagcatgatcaccaccacaa

SEQ ID NO: 83 - DNA, 38 bp, Artificial Sequence
  PCR primer
tgtaaaacgacggccagtaagtttggatggatttgcgt

SEQ ID NO: 84 - DNA, 20 bp, Artificial Sequence
  PCR primer
aagaatctctggtggcttgc

SEQ ID NO: 85 - DNA, 38 bp, Artificial Sequence
  PCR primer
tgtaaaacgacggccagtcaacaacaggagcaccttca

SEQ ID NO: 86 - DNA, 20 bp, Artificial Sequence
  PCR primer
ttgtgtaccttccacatccg

SEQ ID NO: 87 - DNA, 38 bp, Artificial Sequence
  PCR primer
tgtaaaacgacggccagtctttctctcatcccaaccca

SEQ ID NO: 88 - DNA, 20 bp, Artificial Sequence
  PCR primer
tgctcagctcatcaccaatc

SEQ ID NO: 89 - DNA, 38 bp, Artificial Sequence
  PCR primer
tgtaaaacgacggccagtgaaatggtgttcaatggcct

SEQ ID NO: 90 - DNA, 20 bp, Artificial Sequence
  PCR primer
cgaaattccaaacacgttca

SEQ ID NO: 91 - DNA, 38 bp, Artificial Sequence
  PCR primer
tgtaaaacgacggccagttcctcttaagcgcatcccta

SEQ ID NO: 92 - DNA, 20 bp, Artificial Sequence
  PCR primer
agtctttgtcctcgctcgtc

SEQ ID NO: 93 - DNA, 38 bp, Artificial Sequence
  PCR primer
tgtaaaacgacggccagtgtggtggagagaaggcagag

SEQ ID NO: 94 - DNA, 20 bp, Artificial Sequence
  PCR primer
tccagtgcctgtttcagttg

SEQ ID NO: 95 - DNA, 38 bp, Artificial Sequence
  PCR primer
tgtaaaacgacggccagtctccgtatgcaagtttggct

SEQ ID NO: 96 - DNA, 20 bp, Artificial Sequence
  PCR primer
cgttgtgaaacctgggagat

SEQ ID NO: 97 - DNA, 39 bp, Artificial Sequence
  PCR primer
tgtaaaacgacggccagttgaaggcagggagtgtaccta

SEQ ID NO: 98 - DNA, 20 bp, Artificial Sequence
  PCR primer
catcatggcaagacaacgag

SEQ ID NO: 99 - DNA, 39 bp, Artificial Sequence
  PCR primer
tgtaaaacgacggccagtgggcatggatcacagtacaga

SEQ ID NO: 100 - DNA, 20 bp, Artificial Sequence
  PCR primer
ttgagaggctttgctcttgg

SEQ ID NO: 101 - DNA, 38 bp, Artificial Sequence
  PCR primer
tgtaaaacgacggccagttgagtgttaattgggaggca

SEQ ID NO: 102 - DNA, 20 bp, Artificial Sequence
  PCR primer
aggtggtcattcgggtcata

SEQ ID NO: 103 - DNA, 38 bp, Artificial Sequence
  PCR primer
tgtaaaacgacggccagtgcatgcatccaggtcctatt

SEQ ID NO: 104 - DNA, 20 bp, Artificial Sequence
  PCR primer
ctataagcttcgcacctgcc

SEQ ID NO: 105 - DNA, 38 bp, Artificial Sequence
  PCR primer
tgtaaaacgacggccagtcggtggacggatcagttagt

SEQ ID NO: 106 - DNA, 20 bp, Artificial Sequence
  PCR primer
ggaaggaggccaagtttgtt

SEQ ID NO: 107 - DNA, 38 bp, Artificial Sequence
  PCR primer
tgtaaaacgacggccagtcgcagcagctatttctaggc

SEQ ID NO: 108 - DNA, 20 bp, Artificial Sequence
  PCR primer
tgctgtgctggctacttcat

SEQ ID NO: 109 - DNA, 38 bp, Artificial Sequence
  PCR primer
tgtaaaacgacggccagtttgactgaggacactttgcg

SEQ ID NO: 110 - DNA, 20 bp, Artificial Sequence
  PCR primer
agcatcttcggcttcattgt

SEQ ID NO: 111 - DNA, 38 bp, Artificial Sequence
  PCR primer
tgtaaaacgacggccagtttcttcggtgtaggtggagc

SEQ ID NO: 112 - DNA, 20 bp, Artificial Sequence
  PCR primer
agactcagcgcaaaggctaa

SEQ ID NO: 113 - DNA, 38 bp, Artificial Sequence
  PCR primer
tgtaaaacgacggccagtatttggccatccagatgttt

SEQ ID NO: 114 - DNA, 19 bp, Artificial Sequence
  PCR primer
cattaagctcgcgcaattc

SEQ ID NO: 115 - DNA, 38 bp, Artificial Sequence
  PCR primer
tgtaaaacgacggccagtcgaggtctacgcacaaatga

SEQ ID NO: 116 - DNA, 20 bp, Artificial Sequence
  PCR primer
agaattcggtaggttgacgg

SEQ ID NO: 117 - DNA, 38 bp, Artificial Sequence
  PCR primer
tgtaaaacgacggccagtgcagcctcagttgtctttcc

SEQ ID NO: 118 - DNA, 20 bp, Artificial Sequence
  PCR primer
acttccggcctttccatagt

SEQ ID NO: 119 - DNA, 38 bp, Artificial Sequence
  PCR primer
tgtaaaacgacggccagtcaagcccgagtaggaatcag

SEQ ID NO: 120 - DNA, 20 bp, Artificial Sequence
  PCR primer
ccagcaccaatcagttcaaa

SEQ ID NO: 121 - DNA, 38 bp, Artificial Sequence
  PCR primer
tgtaaaacgacggccagtacatcagaagacctgcaccc

SEQ ID NO: 122 - DNA, 20 bp, Artificial Sequence
  PCR primer
tgagcgtccctggaaactac

SEQ ID NO: 123 - DNA, 38 bp, Artificial Sequence
  PCR primer
tgtaaaacgacggccagttcgagaaacaaatgtcccgt

SEQ ID NO: 124 - DNA, 20 bp, Artificial Sequence
  PCR primer
atgttcaaatatcgcgcaaa

SEQ ID NO: 125 - DNA, 38 bp, Artificial Sequence
  PCR primer
tgtaaaacgacggccagtcacctccttatatgcgctgg

SEQ ID NO: 126 - DNA, 20 bp, Artificial Sequence
  PCR primer
cacgtatagatggtgcacgg

SEQ ID NO: 127 - DNA, 38 bp, Artificial Sequence
  PCR primer
tgtaaaacgacggccagtttggagtaaggcgtagggaa

SEQ ID NO: 128 - DNA, 20 bp, Artificial Sequence
  PCR primer
gcctcagctggagactgatt

SEQ ID NO: 129 - DNA, 38 bp, Artificial Sequence
  PCR primer
tgtaaaacgacggccagtttagccaaccgtaacgaacc

SEQ ID NO: 130 - DNA, 20 bp, Artificial Sequence
  PCR primer
tcgattgattgaggaagcgt

SEQ ID NO: 131 - DNA, 38 bp, Artificial Sequence
  PCR primer
tgtaaaacgacggccagtagccgcctcctctgactatt

SEQ ID NO: 132 - DNA, 20 bp, Artificial Sequence
  PCR primer
tgctgtgatgattcggtgat

SEQ ID NO: 133 - DNA, 38 bp, Artificial Sequence
  PCR primer
tgtaaaacgacggccagttgccgcttaggaagatttgt

SEQ ID NO: 134 - DNA, 20 bp, Artificial Sequence
  PCR primer
ccatgaacatttgctggatg

SEQ ID NO: 135 - DNA, 38 bp, Artificial Sequence
  PCR primer
tgtaaaacgacggccagtcgtcactcggatccatctct

SEQ ID NO: 136 - DNA, 20 bp, Artificial Sequence
  PCR primer
cgaaccaaacgaaggtgagt

SEQ ID NO: 137 - DNA, 38 bp, Artificial Sequence
  PCR primer
tgtaaaacgacggccagtggagaacttggaggacgaga

SEQ ID NO: 138 - DNA, 20 bp, Artificial Sequence
  PCR primer
tgatgaaaccacatgcttgg

SEQ ID NO: 139 - DNA, 38 bp, Artificial Sequence
  PCR primer
tgtaaaacgacggccagtatggtgaaggcagatggaac

SEQ ID NO: 140 - DNA, 20 bp, Artificial Sequence
  PCR primer
tgacccttcttgaggtctgg

SEQ ID NO: 141 - DNA, 38 bp, Artificial Sequence
  PCR primer
tgtaaaacgacggccagtccacagtgagacgtacacgc

SEQ ID NO: 142 - DNA, 20 bp, Artificial Sequence
  PCR primer
acgctcccttgttggaaata

SEQ ID NO: 143 - DNA, 38 bp, Artificial Sequence
  PCR primer
tgtaaaacgacggccagtgcgaacttggccataaatct

SEQ ID NO: 144 - DNA, 20 bp, Artificial Sequence
  PCR primer
ggatgagcctgagctacgaa

SEQ ID NO: 145 - DNA, 38 bp, Artificial Sequence
  PCR primer
tgtaaaacgacggccagtccggaatcagttcaaaccat

SEQ ID NO: 146 - DNA, 20 bp, Artificial Sequence
  PCR primer
gccaagctatttgccacttc

SEQ ID NO: 147 - DNA, 38 bp, Artificial Sequence
  PCR primer
tgtaaaacgacggccagtcccgagttacatcgaatggt

SEQ ID NO: 148 - DNA, 20 bp, Artificial Sequence
  PCR primer
caagttgcgcagattcttga

SEQ ID NO: 149 - DNA, 38 bp, Artificial Sequence
  PCR primer
tgtaaaacgacggccagtagttgcaagttgtgtgcgaa

SEQ ID NO: 150 - DNA, 20 bp, Artificial Sequence
  PCR primer
cgacatacagtaaagcgcca

SEQ ID NO: 151 - DNA, 38 bp, Artificial Sequence
  PCR primer
tgtaaaacgacggccagtacttggcgttcttgtggaag

SEQ ID NO: 152 - DNA, 20 bp, Artificial Sequence
  PCR primer
agctttgcaagtttgtgctg

SEQ ID NO: 153 - DNA, 38 bp, Artificial Sequence
  PCR primer
tgtaaaacgacggccagtaacatggagcgatgctgata

SEQ ID NO: 154 - DNA, 20 bp, Artificial Sequence
  PCR primer
ccatccctttgttctcgatg

SEQ ID NO: 155 - DNA, 38 bp, Artificial Sequence
  PCR primer
tgtaaaacgacggccagttgtttgcggttgaagacaag

SEQ ID NO: 156 - DNA, 21 bp, Artificial Sequence
  PCR primer
ctgatgacaccactggaacct

SEQ ID NO: 157 - DNA, 38 bp, Artificial Sequence
  PCR primer
tgtaaaacgacggccagtttgtatgggcgcactatgaa

SEQ ID NO: 158 - DNA, 20 bp, Artificial Sequence
  PCR primer
tgcccttctttagccaagtc

SEQ ID NO: 159 - DNA, 38 bp, Artificial Sequence
  PCR primer
tgtaaaacgacggccagtgaagtagctccgtgtgaggc

SEQ ID NO: 160 - DNA, 20 bp, Artificial Sequence
  PCR primer
agcctcgtctcatagttggc

SEQ ID NO: 161 - DNA, 38 bp, Artificial Sequence
  PCR primer
tgtaaaacgacggccagtgtcgtcctatgatgccacct

SEQ ID NO: 162 - DNA, 20 bp, Artificial Sequence
  PCR primer
tcgcagcattgtattgtggt

SEQ ID NO: 163 - DNA, 38 bp, Artificial Sequence
  PCR primer
tgtaaaacgacggccagtagcaaggaagccaagtatcg

SEQ ID NO: 164 - DNA, 20 bp, Artificial Sequence
  PCR primer
ttattcccgcgattccatta

SEQ ID NO: 165 - DNA, 38 bp, Artificial Sequence
  PCR primer
tgtaaaacgacggccagtgcatcatacgttgagcacca

SEQ ID NO: 166 - DNA, 20 bp, Artificial Sequence
  PCR primer
gccaaactctgccatttgac

SEQ ID NO: 167 - DNA, 38 bp, Artificial Sequence
  PCR primer
tgtaaaacgacggccagttgagggcttaacttcgttgg

SEQ ID NO: 168 - DNA, 20 bp, Artificial Sequence
  PCR primer
cgtttggaaggtcgaacact

SEQ ID NO: 169 - DNA, 38 bp, Artificial Sequence
  PCR primer
tgtaaaacgacggccagttgatcaacgacgatgcattt

SEQ ID NO: 170 - DNA, 20 bp, Artificial Sequence
  PCR primer
aagctttcccgtcttggttt

SEQ ID NO: 171 - DNA, 38 bp, Artificial Sequence
  PCR primer
tgtaaaacgacggccagttggcctcggttatgttcttc

SEQ ID NO: 172 - DNA, 20 bp, Artificial Sequence
  PCR primer
caaacgagagtgccagtcag

SEQ ID NO: 173 - DNA, 38 bp, Artificial Sequence
  PCR primer
tgtaaaacgacggccagtggtgagtgaacggtgtgaga

SEQ ID NO: 174 - DNA, 20 bp, Artificial Sequence
  PCR primer
ccatctgcttaaaccaaggc

SEQ ID NO: 175 - DNA, 38 bp, Artificial Sequence
  PCR primer
tgtaaaacgacggccagttccaacagagaggtgaaggg

SEQ ID NO: 176 - DNA, 20 bp, Artificial Sequence
  PCR primer
caggccagtagggcaatagt

SEQ ID NO: 177 - DNA, 39 bp, Artificial Sequence
  PCR primer
tgtaaaacgacggccagttgacgaggctgatgactcttt

SEQ ID NO: 178 - DNA, 20 bp, Artificial Sequence
  PCR primer
ttcctggcgcagagtctaat

SEQ ID NO: 179 - DNA, 38 bp, Artificial Sequence
  PCR primer
tgtaaaacgacggccagtcgtcgggatattggaaagag

SEQ ID NO: 180 - DNA, 20 bp, Artificial Sequence
  PCR primer
gatcctccatgactaccgct

SEQ ID NO: 181 - DNA, 38 bp, Artificial Sequence
  PCR primer
tgtaaaacgacggccagtcaacactgccacaatcaacc

SEQ ID NO: 182 - DNA, 20 bp, Artificial Sequence
  PCR primer
aggcgacatgtaaccaacaa

SEQ ID NO: 183 - DNA, 37 bp, Artificial Sequence
  PCR primer
tgtaaaacgacggccagtttggtgttaggaagcgtgc

SEQ ID NO: 184 - DNA, 20 bp, Artificial Sequence
  PCR primer
ttgcatgaccctcagcatag

SEQ ID NO: 185 - DNA, 38 bp, Artificial Sequence
  PCR primer
tgtaaaacgacggccagtgaagaacgttacgcctggaa

SEQ ID NO: 186 - DNA, 20 bp, Artificial Sequence
  PCR primer
aaatgggccgtatccttagc

SEQ ID NO: 187 - DNA, 38 bp, Artificial Sequence
  PCR primer
tgtaaaacgacggccagtatttgttggttccctgtcgt

SEQ ID NO: 188 - DNA, 20 bp, Artificial Sequence
  PCR primer
aacccaggtttagccacaga

SEQ ID NO: 189 - DNA, 38 bp, Artificial Sequence
  PCR primer
tgtaaaacgacggccagtcgaactctccgttccgtatg

SEQ ID NO: 190 - DNA, 20 bp, Artificial Sequence
  PCR primer
atttggtgccttcaaaccag

SEQ ID NO: 191 - DNA, 38 bp, Artificial Sequence
  PCR primer
tgtaaaacgacggccagtgttgctgcgctacacatcac

SEQ ID NO: 192 - DNA, 20 bp, Artificial Sequence
  PCR primer
gataaccgcttggcaacact

(19) United States
(12) Patent Application Publication
(10) Pub. No.: US 2010/0235946 A1
(43) Pub. Date: Sep. 16, 2010

(54) PLANT TRANSCRIPTIONAL FACTORS AS MOLECULAR MARKERS

(76) Inventors: Yuanhong Han, Ardmore, OK (US); Dong-Man Khu, Ardmore, OK (US); Maria J. Monteros, Ardmore, OK (US); Ivone Torres-Jerez, Ardmore, OK (US); Michael Udvardi, Ardmore, OK (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(21) Appl. No.: 12/635,589

(22) Filed: Dec. 10, 2009

Related U.S. Application Data
(60) Provisional application No. 61/121,483, filed on Dec. 10, 2008.

(51) Int. Cl. A01H 1/06; C12Q 1/68; C07H 21/04; G01N 27/26

(52) U.S. Cl. 800/279; 435/6; 536/23.1; 800/278; 800/289; 204/461

(57) ABSTRACT

The present invention discloses methods for identification and use of nucleotide sequences associated with loci encoding plant transcription factors as markers for genetic mapping and breeding in plant species including legume species such as Medicago spp., Lotus japonicus, Glycine max, Pisum sativum, Phaseolus vulgaris, Vigna radiata, V. unguiculata, Trifolium spp., and Lupinus albus.

### CROSS-REFERENCE TO RELATED APPLICATIONS

This application claims the benefit of priority of U.S. Provisional Application Ser. No. 61/121,483, filed on Dec. 10, 2008, the disclosure of which is incorporated herein by reference in its entirety.

### BACKGROUND OF THE INVENTION

1. Field of the Invention

The present invention relates generally to plant genetics. More specifically, the invention relates to identification and use of loci encoding plant transcription factors as markers for genetic mapping and breeding in plant species including legume species.

2. Description of Related Art

Molecular markers have been used to determine genetic relatedness between plant materials, to assist in the identification of novel sources of genetic variation, to confirm the pedigree and identity of new varieties, to locate quantitative trait loci (QTL) and genes of interest, and for marker-assisted breeding. Markers have also been used to investigate genes and gene interactions for a number of quantitative traits in several important crop species. The value and uses of various types of DNA markers have been shaped in large part by contemporary innovations in marker technologies that increased throughput and reduced costs per data point. However, the major constraint in using molecular markers has been the cost and effort required to develop them. Traditionally, molecular markers, such as microsatellites, need to be cloned and sequenced for each target species in a process which can be laborious, expensive, and time consuming. A more widespread use of markers would be facilitated if they were transferable across multiple species, which would reduce the need to develop species-specific markers. In general, the extent of marker transferability between species depends on the evolutionary rate of the flanking sequences as well as of the target sequences themselves. The identification of conserved priming sites among multiple taxa can be used to facilitate the transfer of information from models to crops. A survey of microsatellite marker transferability in large plant families indicates that most markers work well within the genus of origin and closely related taxa, but less so as the phylogenetic distance increases, and may not work at all in species from other genera. Hence, it appears that the transferability of current molecular markers across genus borders is limited.

Development of a comprehensive resource of plant transcription factors (“TF's”) in model and crop plant species, including legumes, and evaluation of the nucleotide sequences associated with genes encoding such TF's, as molecular markers for comparative genetic mapping across a wide range of plants, such as forage and crop legume species including those with limited genomic resources, would be of great benefit for plant breeders and agriculture in general.

### BRIEF DESCRIPTION OF THE FIGURES

FIG. 1: Phylogenetic relationships between legume species (adapted from Zhu et al., 2005).

FIG. 2A-B: GeneMapper output illustrating different scenarios in PCR amplification products of two representative transcription factors evaluated across multiple legume species. A. TF56E02 produced a single PCR amplicon of the same length (152 bp) in all species. B. TF56C11 produced PCR amplicons of different lengths in each of the legume species in the panel.

### SUMMARY OF THE INVENTION

In one aspect, the invention provides a method for detecting the location of a locus of interest in a plant comprising: (a) identifying a sequence from a first plant transcription factor gene of a plant of a first plant species, wherein the transcription factor gene is genetically linked to a locus of interest in said plant; and (b) detecting the presence of a sequence from an orthologous plant transcription factor gene in a plant of a second plant species; wherein the orthologous plant transcription factor gene is genetically linked to an orthologous locus of interest in the plant of the second plant species, whereby the presence of the orthologous plant transcription factor gene is indicative of the presence of the orthologous locus of interest in the plant. In one embodiment, identifying a sequence from a first plant transcription factor gene and/or detecting the presence of a sequence from an orthologous plant transcription factor gene comprises detecting the presence of a polymorphism in said first plant transcription factor gene and/or said orthologous plant transcription factor gene.

In certain embodiments, the first and second plant species are legume (Leguminosae) species or grass species. In other embodiments, the first and second plant species are Galegoid legume species. In yet other embodiments, the first and second plant species are Phaseoloid legume species. In still yet other embodiments, the first plant species is a Phaseoloid legume species and the second plant species is a Galegoid legume species. In yet other embodiments, the first plant species is a Galegoid legume species and the second plant species is a Phaseoloid legume species.

In certain embodiments the first and second plant species are selected from members of the group consisting of the tribes Viceae, Trifoleae, Cicereae, Loteae, and Phaseoleae. The first and second plant species may also be selected, in certain embodiments, from the members of the group consisting of the genera Lens, Vicia, Pisum, Melilotus, Trifolium, Medicago, Cicer, Lotus, Phaseolus, Vigna, Glycine, Arachis, and Cajanus. In other embodiments, the first and second plant species are selected from members of the group consisting of the genera Medicago, Lotus, Phaseolus, Glycine, Festuca, Panicum, and Triticum. In particular embodiments the first and second plant species are Medicago sp. or Glycine sp.

Yet another embodiment of the invention provides a method such as described above, wherein detecting the presence of a plant transcription factor gene or an orthologous plant transcription factor gene comprises a technique selected from the group consisting of: PCR, nucleotide hybridization, single strand conformational polymorphism analysis, denaturing gradient gel electrophoresis, cleavage fragment length polymorphism analysis and/or DNA sequencing.

In certain embodiments, detecting the presence of a plant transcription factor gene in a first plant species and detecting the presence of an orthologous plant transcription factor gene in a second plant species comprises utilizing the same technique for each species. In particular embodiments the technique comprises utilization of a primer pair or a hybridization probe. In more particular embodiments the primer pair or hybridization probe utilized for each plant species comprises the same nucleotide sequence.

Another aspect of the invention provides a method for breeding a plant comprising: (a) identifying a sequence from a first plant transcription factor gene of a plant of a first plant species, wherein the transcription factor gene is genetically linked to a locus of interest in said plant; and (b) detecting the presence of a sequence from an orthologous plant transcription factor gene in a plant of a second plant species; wherein the orthologous plant transcription factor gene is genetically linked to an orthologous locus of interest in the plant of the second plant species, whereby the presence of the orthologous plant transcription factor gene is indicative of the presence of the orthologous locus of interest in the plant into the genome of a plant by performing marker-assisted selection, and introgressing the trait genetically linked to a first or second locus into the genome of a plant by performing marker-assisted selection.

In certain embodiments, marker-assisted selection comprises PCR, nucleotide hybridization, single strand conformational polymorphism analysis, denaturing gradient gel electrophoresis, cleavage fragment length polymorphism analysis and/or DNA sequencing.

In some embodiments the trait is selected from the group consisting of: tolerance to abiotic stress, tolerance to biotic stress, increased yield, increased nodulation, altered oil content, altered protein content, altered flavonoid content, maturity group, and time of flowering. In other embodiments the trait confers increased tolerance to wounding, salt, cold, heat, drought, oxidative stress, aluminum, pest infestation, or pathogen infection.

Another aspect of the invention provides an isolated nucleic acid molecule comprising a sequence selected from the group consisting of SEQ ID NOs:1-192.

Yet another aspect of the invention provides a computer readable data storage medium encoded with computer readable data comprising: one or more nucleotide sequences identified according to the method of claim 1.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention.

The invention provides methods and compositions for genetic mapping in plant species including legume species. Transcription factors (“TF's”) are global regulators of gene expression and represent excellent targets for developing molecular markers which may be used in comparative genetic analyses between multiple crop plant species. The present invention relates to use of sequences associated with genes encoding plant transcription factors for genetic mapping across plant species. PCR amplification of molecular markers allows for developing transcription factor sequences from plants such as Medicago truncatula and other legumes, for use across multiple model and crop plants, and in particular, legume species. Further, the present invention addresses existing gaps in plant and legume comparative genomics by targeting global regulators of gene expression (e.g. transcription factor associated sequences), and also by including white clover, red clover, and alfalfa, which are perennial, tetraploid, and outcrossing legumes, in comparative mapping studies previously dominated by diploid, annual species with a selfing mode of reproduction. The unique features of these species offer opportunities to further understand legume genome structure and evolution, and allow identification of molecular markers with applicability across numerous plant genomes.

In eukaryotic organisms, an integrated regulatory network includes transcription factors, target genes and their relationships. Regulation of gene expression at the transcriptional level influences or controls many of the biological processes in an organism and includes growth and development, metabolic and physiological balance, and responses to the environment (Reichmann et al., 2000). Development is often controlled by transcription factors acting as switches in regulatory cascades. Transcription factors (“TF's”) are defined as proteins that show sequence-specific DNA binding and are capable of activating and/or repressing transcription. Most known transcription factors can be grouped into families according to their DNA binding domain, and putative TF genes are identified based on DNA sequences that encode known DNA-binding domains. Transcription factors regulate the transcription of most, if not all genes. The importance of transcription factors in plant biology is reflected by the fact that approximately 7% of all plant genes encode such proteins (Reichmann et al., 2000). The sequence conservation of these binding domains allowed a genome-wide comparative analysis among three eukaryotic kingdoms including plants, animals, and fungi. Most of the transcription factor families were either shared by the three lineages if they were present in the common ancestor or specific to each lineage if they arose independently following divergence.

Transcription factors are key components in understanding regulation of important plant processes (Kakar, 2008). Transcription factors are involved in abiotic stress responses including drought, freezing, salt and aluminum tolerance (Zhang, et al., 2005; Dai et al., 2007; Iuchi et al., 2007), in plant defense responses (Libault et al., 2007; Raffaele et al., 2008), detoxification and stress responses (Mueller et al., 2008), in the development and differentiation of root nodules (Schauser et al., 1999), and in flowering time (Cai et al., 2007). Over-expression of transcription factor sequences has led to increases in freezing, drought, salt, and soil toxicity stress tolerance (Zhang et al., 2005; Dai et al., 2007; Iuchi et al., 2007; Li et al., 2008). Transcription factors also activate several genes involved in the flavonoid biosynthetic pathway, which contribute to the pigmentation of flowers, leaves, and seeds, and are also involved in signaling between plants and microbes (Deluc et al., 2008). A recently characterized MYB transcription factor provided new evidence for the conserved mechanism in regulation of the flavonoid pathway within the plant kingdom (Ban et al., 2007).

Comparative genome analyses can reveal genetic conservation among the genomes of related species (synteny) and greatly facilitate gene discovery. Synteny refers to a conserved gene order between species revealed by comparative genetic mapping of common DNA markers or in silico mapping of homologous sequences. Based on synteny and other molecular information, molecular markers identified through the evaluation of TF primers developed in certain plant species, such as the model legume M. truncatula, might serve as anchor markers for genetic mapping across species and higher plant taxonomic units. Thus, for instance, TF-associated markers identified from one legume species may be applied to genetic mapping in other legume species, to genetic mapping in grasses such as tall fescue, switchgrass, and wheat, and to other plants, and vice-versa.

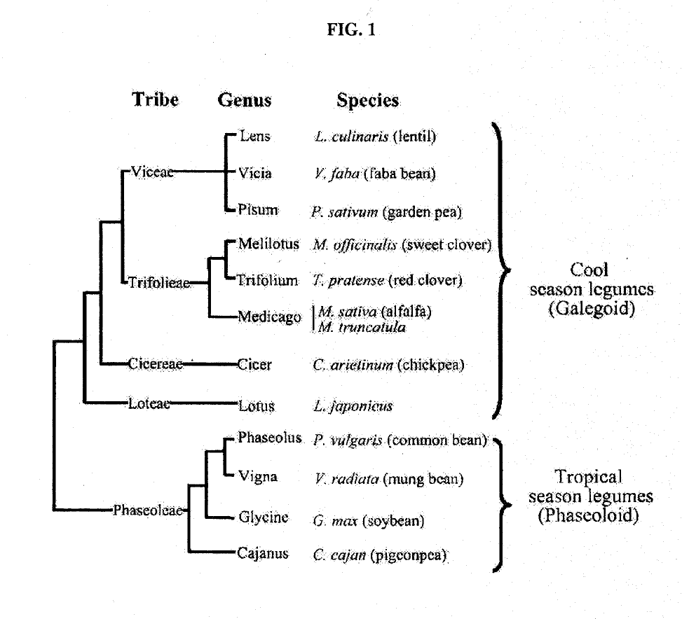

Legumes represent an important component of the world's crop production due to their symbiotic nitrogen fixation capabilities, high protein and oil content, and nutritive value. Traditionally, legume species have been studied separately and genomic resources have been developed independently for each crop. Most important crop legumes including soybean (Glycine max), chickpea (Cicer arietinum), peas (Pisum spp.), beans (Vicia spp.), lentils (Lens culinaris), alfalfa (M. sativa), peanut (Arachis hypogaea), and clovers (Trifolium spp.), occur in the Phaseoloid and Galegoid clades (FIG. 1). Although genomic resources are available in some model plant species such as the legumes M. truncatula, Lotus japonicus, and soybean, other legume species including alfalfa and white clover have lagged behind in genomic resource development and support. Further, despite their close phylogenetic relationships, crop legumes and model legumes differ in genome size, chromosome number, ploidy level, and self-compatibility (Zhu et al., 2005) (Table 1).

TABLE 1
Chromosome number and genome size of selected
model and crop legumes (from Zhu et al., 2005).

| | | Chromosome | Genome size | Reproductive |
|---|---|---|---|---|
| Species | Common name | No. | (Mb/1C) | system |
| Medicago truncatula | Barrel medic | 2n = 2x = 16 | 466 | Selfing |
| Medicago sativa | Alfalfa | 2n = 4x = 32 | 1,715 | Outcrossing |
| Trifolium repens | White clover | 2n = 4x = 32 | 956 | Outcrossing |
| Lotus japonicus | Lotus | 2n = 2x = 16 | 466 | Selfing |
| Glycine max | Soybean | 2n = 4x = 40 | 1,103 | Selfing |
| Phaseolus vulgaris | Common bean | 2n = 2x = 22 | 588 | Selfing |
| Arachis hypogaea | Peanut | 2n = 4x = 40 | | |

Initial evaluations of cross-species amplification using molecular markers suggested that successful cross-species amplification of simple sequence repeats (SSRs) in plants was largely restricted to congeners or closely related genera (Peakall et al., 1998). Comparative studies in legumes with a limited number of molecular markers has included comparisons among Medicago species including alfalfa (M. sativa), white clover (Trifolium repens), red clover (Trifolium pratense), Subterranean clover (Trifolium subterraneum), L. japonicus, soybean (G. max), pea (P. sativum), mung bean (V. radiata), common bean (P. vulgaris), chickpea (C. arietinum L.), peanut (Arachis hypogaea), and lupin (Lupinus angustifolius). Such studies are of use in developing and expanding genetic maps in the studied species, genera, and families. However none of these studies focused on using sequences associated with plant transcription factors as targets for identification of molecular marker sequences with applicability toward multiple crop and model plant or legume species.

The conserved nature of genetic networks across species and the ability to transfer knowledge from one species to another via comparative genomics and subsequent marker-assisted breeding, and by direct genetic engineering, may lead to potential major innovations in crop improvement, for instance by transferring agriculturally-relevant information from one species to another. In legumes, it has been demonstrated that resistance gene homologs between M. truncatula and both pea and soybean occupy syntenic positions. Identification of plant genes that have remained relatively stable in sequence and copy number since the radiation of flowering plants from their last common ancestors may allow identification of additional molecular markers particularly useful for comparative genome analyses between multiple plant families, clades, tribes, genera, and species. Thus expanding the search for molecular markers to genomic regions associated with various traits of agronomic significance, in particular by utilizing sequences associated with plant transcriptional factors, may facilitate molecular breeding in a wider range of plant species, including legume species. Integrating genomics information from model and crop legumes has immediate applications including the use of marker-assisted selection and breeding to develop enhanced legume cultivars.

M. truncatula and L. japonicus have been selected as initial model species for legumes, in particular Galegoid (cool season) legumes, and soybean has been selected as a representative species for the Phaseoloid (tropical season) legumes. Genome sequencing efforts in M. truncatula and L. japonicus, as well as soybean and common bean can also be used to facilitate cross-species comparisons between model and crop legumes. For instance, primers producing PCR amplicons in alfalfa (M. sativa L.) may be used to further evaluate amplification in a panel consisting of model (e.g. M. truncatula, Lotus japonicus) and crop legumes (e.g. Glycine max L., Pisum sativum L., Phaseolus vulgaris L., Vigna radiata L., V. unguiculata L., M. sativa L., Trifolium repens L., T. subterraneum, T. pratense L., A. hypogaea, and Lupinus albus L.), among others, that include parents of existing mapping populations. Amplification may also be evaluated in other plants, both dicots and monocots, including tall fescue, switchgrass, and wheat, among others. Amplification, size polymorphism, and sequence variation, among other polymorphic parameters, may be evaluated. The present invention allows for development of a comprehensive resource of global regulators of gene expression, identification of anchor markers which can be used in multiple species for basic and applied genetic studies, and the establishment of a comparative mapping framework that allows transfer of information from model plants to crop plants and vice-versa, including less-well characterized legume species.

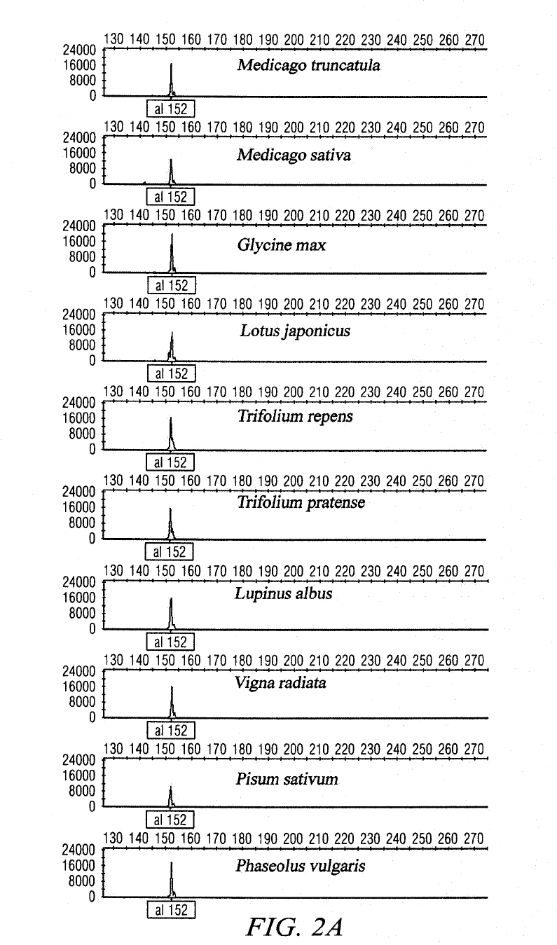

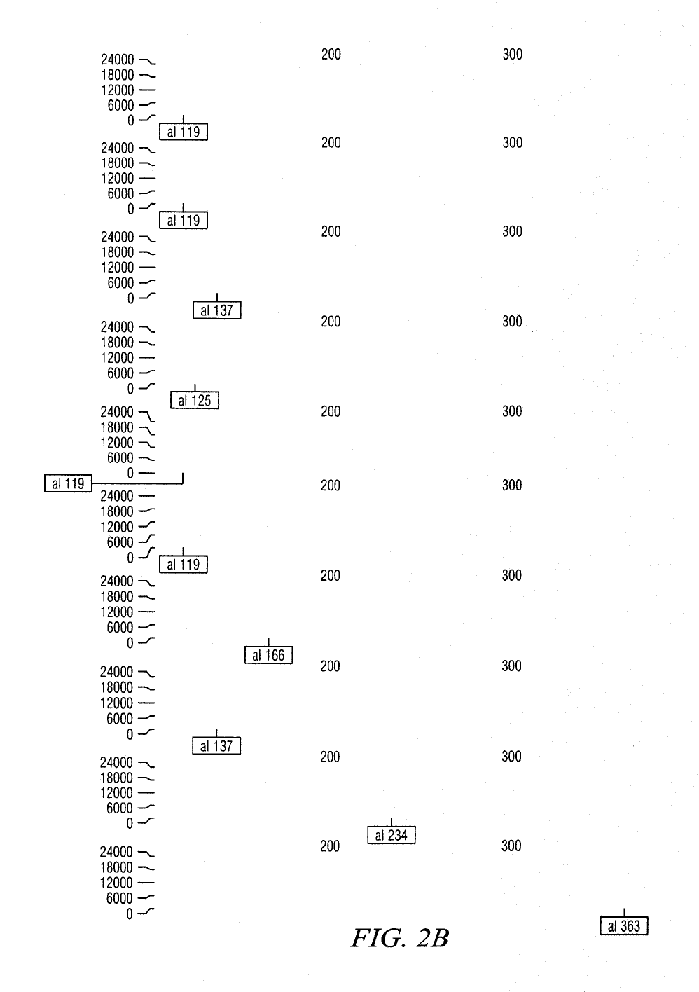

Primers that amplify in only a few species have value in that they can be used to increase the density of molecular markers in existing linkage maps, and when combined with phenotypic data from large mapping populations, can enhance the resolution of future QTL mapping studies for key traits. The availability of anchor markers based on transcription factors designed for gene expression profiling offers a unique opportunity to assess variation both in sequences and in the expression levels of these master regulators across multiple species. Gene expression levels can be treated as expression quantitative trait loci (eQTL) and have been mapped in different species (Morley et al., 2004; West et al., 2007). This approach is robust enough to identify markers associated with both trans and cis-acting factors that could be used in marker-assisted mapping studies, and applied to plant breeding. Transcript profiling can also be used to uncover the function of TF genes/proteins by revealing where and when in a plant these TF genes are expressed. Such TF-associated anchor markers may then be linked to functional information and tissue expression through the M. truncatula Gene Atlas (Benedito et al., 2008). For example, a BLAST query of the 152 by sequence amplified with TF56E02 (FIG. 2) against the M. truncatula Gene Atlas indicates that this TF is highly expressed in flowers and pods and does not appear to be legume specific (data not shown).

The following TF gene-associated primers are provided in Table 2 (SEQ ID NOs:1-192):

TABLE 2
List of TF-associated primers.

| | | |
|---|---|---|
| Primer Name | Forward-Primer (5′ to 3′) | Reverse-Primer (5′ to 3′) |

| | | |
|---|---|---|
| MTTF001 | TGTAAAACGACGGCCAGTTTGTCCATAATCTCTGGTGCC | TCACTTGGCCACATGTCTCT |
| | (SEQ ID NO: 1) | (SEQ ID NO: 2) |
| MTTF002 | TGTAAAACGACGGCCAGTGGGTAGGATCCCAACTAGAGC | ACCAAACCTTAGAGGCCACC |
| | (SEQ ID NO: 3) | (SEQ ID NO: 4) |
| MTTF003 | TGTAAAACGACGGCCAGTGCAAATGCAAATCCTCCAAT | ATCCCAGTTCTGCACAATCC |
| | (SEQ ID NO: 5) | (SEQ ID NO: 6) |
| MTTF004 | TGTAAAACGACGGCCAGTAGCGACCAGAAATACCTCCA | GCTGCCTCAGAGTCTCCTTC |
| | (SEQ ID NO: 7) | (SEQ ID NO: 8) |
| MTTF005 | TGTAAAACGACGGCCAGTGAGGATGTTGCTTGTGATGC | TTTCTGGAAATGTTGCCCTT |
| | (SEQ ID NO: 9) | (SEQ ID NO: 10) |
| MTTF006 | TGTAAAACGACGGCCAGTACCTCCCTGGTAACCCAGAC | TTGAAACCCTTTGTTGCAGA |
| | (SEQ ID NO: 11) | (SEQ ID NO: 12) |
| MTTF007 | TGTAAAACGACGGCCAGTCGACAAAGAAACGGGAAGAG | CGACAAGGGCTGGATTTAGA |
| | (SEQ ID NO: 13) | (SEQ ID NO: 14) |
| MTTF008 | TGTAAAACGACGGCCAGTCGAGGAGGGACAACATTCAT | CAGCATGGGAGCTACAAACA |
| | (SEQ ID NO: 15) | (SEQ ID NO: 16) |
| MTTF009 | TGTAAAACGACGGCCAGTATGGGTTGCAGAAGAGGATG | TTGCCATATACTCCCATGTCC |
| | (SEQ ID NO: 17) | (SEQ ID NO: 18) |
| MTTF010 | TGTAAAACGACGGCCAGTAGCAGCAACAACATTAGGCA | GAATTGCATCTGAAGGAGGG |
| | (SEQ ID NO: 19) | (SEQ ID NO: 20) |
| MTTF011 | TGTAAAACGACGGCCAGTTCATCATAACGGAAGGTGGG | AGCTGCCATGTCATAAGCTGT |
| | (SEQ ID NO: 21) | (SEQ ID NO: 22) |
| MTTF012 | TGTAAAACGACGGCCAGTCGCTAGGGATTGTGATCGTT | GTTGTTGTTACCGCCTCCAC |
| | (SEQ ID NO: 23) | (SEQ ID NO: 24) |
| MTTF013 | TGTAAAACGACGGCCAGTTCAGGCATTCCCTTCAAAGT | CGTGAAAGTGAAGCGACCTA |
| | (SEQ ID NO: 25) | (SEQ ID NO: 26) |
| MTTF014 | TGTAAAACGACGGCCAGTGGTGGAAGGAAGTGCAAGAA | GCCCAAATAAACCATGAGGA |
| | (SEQ ID NO: 27) | (SEQ ID NO: 28) |
| MTTF015 | TGTAAAACGACGGCCAGTATCCATGCCAGATTCTCCAC | AGCCATTTCTACGCTTGCAG |
| | (SEQ ID NO: 29) | (SEQ ID NO: 30) |
| MTTF016 | TGTAAAACGACGGCCAGTTCCACGACCTTCAACAACAA | GGCAGAAGAGATGATAGCCG |
| | (SEQ ID NO: 31) | (SEQ ID NO: 32) |
| MTTF017 | TGTAAAACGACGGCCAGTTGCCGAGTGCTGATTCTATG | GAATTTGCATTCCTTGGTGC |
| | (SEQ ID NO: 33) | (SEQ ID NO: 34) |
| MTTF018 | TGTAAAACGACGGCCAGTGCTGGACTTGAGAGGTGTGG | TGATGACCACCTGTTGCCTA |
| | (SEQ ID NO: 35) | (SEQ ID NO: 36) |
| MTTF019 | TGTAAAACGACGGCCAGTTGAGAAGCTCCATCAAGGGT | CGATTCAAATGGTCCTTTCTTC |
| | (SEQ ID NO: 37) | (SEQ ID NO: 38) |
| MTTF020 | TGTAAAACGACGGCCAGTAGGTGAAGGTTCTTGAGGAGG | CGTCAAAGGGATCACCAGAT |
| | (SEQ ID NO: 39) | (SEQ ID NO: 40) |
| MTTF021 | TGTAAAACGACGGCCAGTGTTCCGGGTACAAAGCATGT | CCAAGGTGAGACACTCGGTC |
| | (SEQ ID NO: 41) | (SEQ ID NO: 42) |
| MTTF022 | TGTAAAACGACGGCCAGTAACAGAGACTGCAACAGCCA | AGCGTAAGTTCCAAGCCAGA |
| | (SEQ ID NO: 43) | (SEQ ID NO: 44) |
| MTTF023 | TGTAAAACGACGGCCAGTTATCGACCCAAATGCAAACA | ACAGCCTTTACGCATCCAAA |
| | (SEQ ID NO: 45) | (SEQ ID NO: 46) |
| MTTF024 | TGTAAAACGACGGCCAGTTCTAAGGCAGTCCTTGTGGG | TTGAGTTGCCATCAGGTTCA |
| | (SEQ ID NO: 47) | (SEQ ID NO: 48) |
| MTTF025 | TGTAAAACGACGGCCAGTTGGGATCAGACAGTCCACAA | GGAACAGAGCCAGAACGGTA |
| | (SEQ ID NO: 49) | (SEQ ID NO: 50) |
| MTTF026 | TGTAAAACGACGGCCAGTGGCCATCATCACAAGGAGTT | TCATGCCTTTGCATCTTCAG |
| | (SEQ ID NO: 51) | (SEQ ID NO: 52) |
| MTTF027 | TGTAAAACGACGGCCAGTCATGCCAGGATCCATTAACC | CACTGAGTCCTCCTCCTGCT |
| | (SEQ ID NO: 53) | (SEQ ID NO: 54) |
| MTTF028 | TGTAAAACGACGGCCAGTAAACGTTGGAACAAGTTGGG | AGCATTTGTTTGGAAGTGGG |
| | (SEQ ID NO: 55) | (SEQ ID NO: 56) |
| MTTF029 | TGTAAAACGACGGCCAGTCGTAGGGATGGAGACAATGAG | AATGTAGCTGGTGGTGGCAT |
| | (SEQ ID NO: 57) | (SEQ ID NO: 58) |
| MTTF030 | TGTAAAACGACGGCCAGTTTGTGTGCGTTGGTCAAGAT | ACGCTTGAGTTCGGCAATAG |
| | (SEQ ID NO: 59) | (SEQ ID NO: 60) |
| MTTF031 | TGTAAAACGACGGCCAGTTCGGGAGCTGGAGTAAGAAA | GGTAATTCAGGATCGGGTCA |
| | (SEQ ID NO: 61) | (SEQ ID NO: 62) |
| MTTF032 | TGTAAAACGACGGCCAGTTGCTGTCAAAGGTGATTGGA | ATCGAGGAAAGACGACGATG |
| | (SEQ ID NO: 63) | (SEQ ID NO: 64) |
| MTTF033 | TGTAAAACGACGGCCAGTGAGTCTAACACAGCCGCACA | CCCTTCACTTCCTGATTCCA |
| | (SEQ ID NO: 65) | (SEQ ID NO: 66) |
| MTTF034 | TGTAAAACGACGGCCAGTTCCGACAACAATTCGAACAC | GTCCTCAATGGCAACATCCT |
| | (SEQ ID NO: 67) | (SEQ ID NO: 68) |
| MTTF035 | TGTAAAACGACGGCCAGTCCAGTGAACAAGCCTGGAAT | CAAATCGGAAGCTCAGAAGG |
| | (SEQ ID NO: 69) | (SEQ ID NO: 70) |
| MTTF036 | TGTAAAACGACGGCCAGTTCATGCAAACTTCTGCTGCT | CCACTGTGATGGCTGAGGTA |
| | (SEQ ID NO: 71) | (SEQ ID NO: 72) |
| MTTF037 | TGTAAAACGACGGCCAGTATTCTTGATGCACCTCCCAC | GCCATATTTGAGTTCCCAGC |
| | (SEQ ID NO: 73) | (SEQ ID NO: 74) |
| MTTF038 | TGTAAAACGACGGCCAGTACAACCACCAATGATGACGA | ATGCAACTTCCCATACCAGC |
| | (SEQ ID NO: 75) | (SEQ ID NO: 76) |
| MTTF039 | TGTAAAACGACGGCCAGTTGAAATTGAAAGGCCACCAT | TTCACCGGGAAGAAGTGAAC |
| | (SEQ ID NO: 77) | (SEQ ID NO: 78) |
| MTTF040 | TGTAAAACGACGGCCAGTTTGGATCTCCTCTGATCCTGA | CTTACCTTTCTTCCCGTCCC |
| | (SEQ ID NO: 79) | (SEQ ID NO: 80) |
| MTTF041 | TGTAAAACGACGGCCAGTTCTTTGTCACCAGACGCAAC | GAGCATGATCACCACCACAA |
| | (SEQ ID NO: 81) | (SEQ ID NO: 82) |
| MTTF042 | TGTAAAACGACGGCCAGTAAGTTTGGATGGATTTGCGT | AAGAATCTCTGGTGGCTTGC |
| | (SEQ ID NO: 83) | (SEQ ID NO: 84) |
| MTTF043 | TGTAAAACGACGGCCAGTCAACAACAGGAGCACCTTCA | TTGTGTACCTTCCACATCCG |
| | (SEQ ID NO: 85) | (SEQ ID NO: 86) |
| MTTF044 | TGTAAAACGACGGCCAGTCTTTCTCTCATCCCAACCCA | TGCTCAGCTCATCACCAATC |
| | (SEQ ID NO: 87) | (SEQ ID NO: 88) |
| MTTF045 | TGTAAAACGACGGCCAGTGAAATGGTGTTCAATGGCCT | CGAAATTCCAAACACGTTCA |
| | (SEQ ID NO: 89) | (SEQ ID NO: 90) |
| MTTF046 | TGTAAAACGACGGCCAGTTCCTCTTAAGCGCATCCCTA | AGTCTTTGTCCTCGCTCGTC |
| | (SEQ ID NO: 91) | (SEQ ID NO: 92) |
| MTTF047 | TGTAAAACGACGGCCAGTGTGGTGGAGAGAAGGCAGAG | TCCAGTGCCTGTTTCAGTTG |
| | (SEQ ID NO: 93) | (SEQ ID NO: 94) |
| MTTF048 | TGTAAAACGACGGCCAGTCTCCGTATGCAAGTTTGGCT | CGTTGTGAAACCTGGGAGAT |
| | (SEQ ID NO: 95) | (SEQ ID NO: 96) |
| MTTF049 | TGTAAAACGACGGCCAGTTGAAGGCAGGGAGTGTACCTA | CATCATGGCAAGACAACGAG |
| | (SEQ ID NO: 97) | (SEQ ID NO: 98) |
| MTTF050 | TGTAAAACGACGGCCAGTGGGCATGGATCACAGTACAGA | TTGAGAGGCTTTGCTCTTGG |
| | (SEQ ID NO: 99) | (SEQ ID NO: 100) |
| MTTF051 | TGTAAAACGACGGCCAGTTGAGTGTTAATTGGGAGGCA | AGGTGGTCATTCGGGTCATA |
| | (SEQ ID NO: 101) | (SEQ ID NO: 102) |
| MTTF052 | TGTAAAACGACGGCCAGTGCATGCATCCAGGTCCTATT | CTATAAGCTTCGCACCTGCC |
| | (SEQ ID NO: 103) | (SEQ ID NO: 104) |
| MTTF053 | TGTAAAACGACGGCCAGTCGGTGGACGGATCAGTTAGT | GGAAGGAGGCCAAGTTTGTT |
| | (SEQ ID NO: 105) | (SEQ ID NO: 106) |
| MTTF054 | TGTAAAACGACGGCCAGTCGCAGCAGCTATTTCTAGGC | TGCTGTGCTGGCTACTTCAT |
| | (SEQ ID NO: 107) | (SEQ ID NO: 108) |
| MTTF055 | TGTAAAACGACGGCCAGTTTGACTGAGGACACTTTGCG | AGCATCTTCGGCTTCATTGT |
| | (SEQ ID NO: 109) | (SEQ ID NO: 110) |
| MTTF056 | TGTAAAACGACGGCCAGTTTCTTCGGTGTAGGTGGAGC | AGACTCAGCGCAAAGGCTAA |
| | (SEQ ID NO: 111) | (SEQ ID NO: 112) |
| MTTF057 | TGTAAAACGACGGCCAGTATTTGGCCATCCAGATGTTT | CATTAAGCTCGCGCAATTC |
| | (SEQ ID NO: 113) | (SEQ ID NO: 114) |
| MTTF058 | TGTAAAACGACGGCCAGTCGAGGTCTACGCACAAATGA | AGAATTCGGTAGGTTGACGG |
| | (SEQ ID NO: 115) | (SEQ ID NO: 116) |
| MTTF059 | TGTAAAACGACGGCCAGTGCAGCCTCAGTTGTCTTTCC | ACTTCCGGCCTTTCCATAGT |
| | (SEQ ID NO: 117) | (SEQ ID NO: 118) |
| MTTF060 | TGTAAAACGACGGCCAGTCAAGCCCGAGTAGGAATCAG | CCAGCACCAATCAGTTCAAA |
| | (SEQ ID NO: 119) | (SEQ ID NO: 120) |
| MTTF061 | TGTAAAACGACGGCCAGTACATCAGAAGACCTGCACCC | TGAGCGTCCCTGGAAACTAC |
| | (SEQ ID NO: 121) | (SEQ ID NO: 122) |
| MTTF062 | TGTAAAACGACGGCCAGTTCGAGAAACAAATGTCCCGT | ATGTTCAAATATCGCGCAAA |
| | (SEQ ID NO: 123) | (SEQ ID NO: 124) |
| MTTF063 | TGTAAAACGACGGCCAGTCACCTCCTTATATGCGCTGG | CACGTATAGATGGTGCACGG |
| | (SEQ ID NO: 125) | (SEQ ID NO: 126) |
| MTTF064 | TGTAAAACGACGGCCAGTTTGGAGTAAGGCGTAGGGAA | GCCTCAGCTGGAGACTGATT |
| | (SEQ ID NO: 127) | (SEQ ID NO: 128) |
| MTTF065 | TGTAAAACGACGGCCAGTTTAGCCAACCGTAACGAACC | TCGATTGATTGAGGAAGCGT |
| | (SEQ ID NO: 129) | (SEQ ID NO: 130) |
| MTTF066 | TGTAAAACGACGGCCAGTAGCCGCCTCCTCTGACTATT | TGCTGTGATGATTCGGTGAT |
| | (SEQ ID NO: 131) | (SEQ ID NO: 132) |
| MTTF067 | TGTAAAACGACGGCCAGTTGCCGCTTAGGAAGATTTGT | CCATGAACATTTGCTGGATG |
| | (SEQ ID NO: 133) | (SEQ ID NO: 134) |
| MTTF068 | TGTAAAACGACGGCCAGTCGTCACTCGGATCCATCTCT | CGAACCAAACGAAGGTGAGT |
| | (SEQ ID NO: 135) | (SEQ ID NO: 136) |
| MTTF069 | TGTAAAACGACGGCCAGTGGAGAACTTGGAGGACGAGA | TGATGAAACCACATGCTTGG |
| | (SEQ ID NO: 137) | (SEQ ID NO: 138) |
| MTTF070 | TGTAAAACGACGGCCAGTATGGTGAAGGCAGATGGAAC | TGACCCTTCTTGAGGTCTGG |
| | (SEQ ID NO: 139) | (SEQ ID NO: 140) |
| MTTF071 | TGTAAAACGACGGCCAGTCCACAGTGAGACGTACACGC | ACGCTCCCTTGTTGGAAATA |
| | (SEQ ID NO: 141) | (SEQ ID NO: 142) |
| MTTF072 | TGTAAAACGACGGCCAGTGCGAACTTGGCCATAAATCT | GGATGAGCCTGAGCTACGAA |
| | (SEQ ID NO: 143) | (SEQ ID NO: 144) |
| MTTF073 | TGTAAAACGACGGCCAGTCCGGAATCAGTTCAAACCAT | GCCAAGCTATTTGCCACTTC |
| | (SEQ ID NO: 145) | (SEQ ID NO: 146) |
| MTTF074 | TGTAAAACGACGGCCAGTCCCGAGTTACATCGAATGGT | CAAGTTGCGCAGATTCTTGA |
| | (SEQ ID NO: 147) | (SEQ ID NO: 148) |
| MTTF075 | TGTAAAACGACGGCCAGTAGTTGCAAGTTGTGTGCGAA | CGACATACAGTAAAGCGCCA |
| | (SEQ ID NO: 149) | (SEQ ID NO: 150) |
| MTTF076 | TGTAAAACGACGGCCAGTACTTGGCGTTCTTGTGGAAG | AGCTTTGCAAGTTTGTGCTG |
| | (SEQ ID NO: 151) | (SEQ ID NO: 152) |
| MTTF077 | TGTAAAACGACGGCCAGTAACATGGAGCGATGCTGATA | CCATCCCTTTGTTCTCGATG |
| | (SEQ ID NO: 153) | (SEQ ID NO: 154) |
| MTTF078 | TGTAAAACGACGGCCAGTTGTTTGCGGTTGAAGACAAG | CTGATGACACCACTGGAACCT |
| | (SEQ ID NO: 155) | (SEQ ID NO: 156) |
| MTTF079 | TGTAAAACGACGGCCAGTTTGTATGGGCGCACTATGAA | TGCCCTTCTTTAGCCAAGTC |
| | (SEQ ID NO: 157) | (SEQ ID NO: 158) |
| MTTF080 | TGTAAAACGACGGCCAGTGAAGTAGCTCCGTGTGAGGC | AGCCTCGTCTCATAGTTGGC |
| | (SEQ ID NO: 159) | (SEQ ID NO: 160) |
| MTTF081 | TGTAAAACGACGGCCAGTGTCGTCCTATGATGCCACCT | TCGCAGCATTGTATTGTGGT |
| | (SEQ ID NO: 161) | (SEQ ID NO: 162) |
| MTTF082 | TGTAAAACGACGGCCAGTAGCAAGGAAGCCAAGTATCG | TTATTCCCGCGATTCCATTA |
| | (SEQ ID NO: 163) | (SEQ ID NO: 164) |
| MTTF083 | TGTAAAACGACGGCCAGTGCATCATACGTTGAGCACCA | GCCAAACTCTGCCATTTGAC |
| | (SEQ ID NO: 165) | (SEQ ID NO: 166) |
| MTTF084 | TGTAAAACGACGGCCAGTTGAGGGCTTAACTTCGTTGG | CGTTTGGAAGGTCGAACACT |
| | (SEQ ID NO: 167) | (SEQ ID NO: 168) |
| MTTF085 | TGTAAAACGACGGCCAGTTGATCAACGACGATGCATTT | AAGCTTTCCCGTCTTGGTTT |
| | (SEQ ID NO: 169) | (SEQ ID NO: 170) |
| MTTF086 | TGTAAAACGACGGCCAGTTGGCCTCGGTTATGTTCTTC | CAAACGAGAGTGCCAGTCAG |
| | (SEQ ID NO: 171) | (SEQ ID NO: 172) |
| MTTF087 | TGTAAAACGACGGCCAGTGGTGAGTGAACGGTGTGAGA | CCATCTGCTTAAACCAAGGC |
| | (SEQ ID NO: 173) | (SEQ ID NO: 174) |
| MTTF088 | TGTAAAACGACGGCCAGTTCCAACAGAGAGGTGAAGGG | CAGGCCAGTAGGGCAATAGT |
| | (SEQ ID NO: 175) | (SEQ ID NO: 176) |
| MTTF089 | TGTAAAACGACGGCCAGTTGACGAGGCTGATGACTCTTT | TTCCTGGCGCAGAGTCTAAT |
| | (SEQ ID NO: 177) | (SEQ ID NO: 178) |
| MTTF090 | TGTAAAACGACGGCCAGTCGTCGGGATATTGGAAAGAG | GATCCTCCATGACTACCGCT |
| | (SEQ ID NO: 179) | (SEQ ID NO: 180) |
| MTTF091 | TGTAAAACGACGGCCAGTCAACACTGCCACAATCAACC | AGGCGACATGTAACCAACAA |
| | (SEQ ID NO: 181) | (SEQ ID NO: 182) |
| MTTF092 | TGTAAAACGACGGCCAGTTTGGTGTTAGGAAGCGTGC | TTGCATGACCCTCAGCATAG |
| | (SEQ ID NO: 183) | (SEQ ID NO: 184) |
| MTTF093 | TGTAAAACGACGGCCAGTGAAGAACGTTACGCCTGGAA | AAATGGGCCGTATCCTTAGC |
| | (SEQ ID NO: 185) | (SEQ ID NO: 186) |
| MTTF094 | TGTAAAACGACGGCCAGTATTTGTTGGTTCCCTGTCGT | AACCCAGGTTTAGCCACAGA |
| | (SEQ ID NO: 187) | (SEQ ID NO: 188) |
| MTTF095 | TGTAAAACGACGGCCAGTCGAACTCTCCGTTCCGTATG | ATTTGGTGCCTTCAAACCAG |
| | (SEQ ID NO: 189) | (SEQ ID NO: 190) |
| MTTF096 | TGTAAAACGACGGCCAGTGTTGCTGCGCTACACATCAC | GATAACCGCTTGGCAACACT |
| | (SEQ ID NO: 191) | (SEQ ID NO: 192) |

### A. Selection of Plants Using Marker-Assisted Selection

A primary motivation for the development of molecular markers in crop species is the potential for increased efficiency in plant breeding through marker-assisted selection (MAS). Procedures for marker assisted selection applicable to the breeding of plants including legumes are well known in the art. Genetic marker alleles (an “allele” is an alternative sequence at a locus) are used to identify plants that contain a desired genotype at multiple loci, and that are expected to transfer the desired genotype, along with a desired phenotype to their progeny. Genetic marker alleles can be used to identify plants that contain the desired genotype at one marker locus, several loci, or a haplotype, and that would be expected to transfer the desired genotype, along with a desired phenotype, to their progeny.

Marker-assisted selection relies on the ability to detect genetic differences between individuals, and marker-assisted breeding comprises assaying genomic DNA for the presence of a genetic marker of interest. A “genetic map” is the representation of the relative position of characterized loci (DNA markers or any other locus for which an allele can be identified) along the chromosomes. The measure of distance is relative to the frequency of crossover events between sister chromatids at meiosis. The genetic differences, or “genetic markers” are then correlated with phenotypic variations using statistical methods. In a preferred case, a single gene encoding a protein responsible for a phenotypic trait is detectable directly by a mutation which results in the variation in phenotype. More commonly, multiple genetic loci each contribute to the observed phenotype.

The presence and/or absence of a particular genetic marker allele in the genome of a plant exhibiting a favorable phenotypic trait is made by correlating the presence of a trait and a genetic marker or markers.

Coinheritance, or genetic linkage, of a particular trait and a marker suggests that they are physically close together on the chromosome. Linkage is determined by analyzing the pattern of inheritance of a gene and a marker in a cross. The unit of recombination is the centimorgan (cM). Two markers are one centimorgan apart if they recombine in meiosis once in every 100 opportunities that they have to do so. The centimorgan is a genetic measure, not a physical one. Those markers located less then 50 cM from a second locus are said to be genetically linked, because they are not inherited independently of one another. Thus, the percent of recombination observed between the loci per generation will be less than 50%. In particular embodiments of the invention, markers may be used located less than about 45, 35, 25, 15, 10, 5, 4, 3, 2, or 1 or less cM apart on a chromosome. In certain embodiments of the invention markers may be used detecting polymorphisms within the contributing loci themselves and thus located at 0 cM respective to the loci.

During meiosis, pairs of homologous chromosomes come together and exchange segments in a process called recombination. The further a marker is from a gene, the more chance there is that there will be recombination between the gene and the marker. In a linkage analysis, the coinheritance of marker and gene or trait are followed in a particular cross. The probability that their observed inheritance pattern could occur by chance alone, i.e., that they are completely unlinked, is calculated. The calculation is then repeated assuming a particular degree of linkage, and the ratio of the two probabilities (no linkage versus a specified degree of linkage) is determined. This ratio expresses the odds for (and against) that degree of linkage, and because the logarithm of the ratio is used, it is known as the logarithm of the odds, e.g. an lod score. A lod score equal to or greater than 3, for example, is taken to confirm that gene and marker are linked. This represents 1000:1 odds that the two loci are linked. Calculations of linkage are greatly facilitated by use of statistical analysis employing programs.

The term “homolog” as used herein refers to a gene related to a second gene by identity of either the DNA sequences or the encoded protein sequences. Genes that are homologs can be genes that are separated by the event of speciation (e.g. an “ortholog”). Genes that are homologs may also be genes separated by the event of genetic duplication (e.g. a “paralog”). Homologs can be from the same or a different organism and may perform the same biological function in either the same or a different organism. When sequence data is available for a particular plant species, orthologous genes are generally identified by sequence similarity analysis, such as a BLAST analysis. Sequences may be assigned as potential orthologs if the best hit sequence from the forward BLAST result retrieves the original query sequence in the reverse BLAST (e.g. Huynen and Bork, 1998; Huynen et al., 2000). Programs for multiple sequence alignment, such as CLUSTAL (Thompson et al., 1994) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Nucleic acid hybridization methods may also be used to find orthologous genes, for instance when sequence data are not available. Degenerate PCR and screening of cDNA or genomic DNA libraries are common methods for finding related gene sequences and are well known in the art (see, e.g., Sambrook et al., 1989).

The genetic linkage of marker molecules can be established by a gene mapping model such as, without limitation, the flanking marker model reported by Lander and Botstein (1989), and interval mapping based on maximum likelihood methods described by Lander and Botstein (1989), and implemented in the software package MAPMAKER/QTL (Lincoln and Lander, 1990). Additional software includes Qgene, Version 2.23 (1996) (Department of Plant Breeding and Biometry, 266 Emerson Hall, Cornell University, Ithaca, N.Y.).

Examples of DNA markers include Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), single feature polymorphisms (SFPs, for example, as described in Borevitz et al. 2003), haplotypes, tag SNPs, Sequence Characterized Amplified Regions (SCARs), alleles of genetic markers, genes, DNA-derived sequences, RNA-derived sequences, promoters, 5′ untranslated regions of genes, 3′ untranslated regions of genes, microRNA, siRNA, quantitative trait loci (QTL), satellite markers, transgenes, mRNA, ds mRNA, transcriptional profiles, and methylation patterns and others known to those skilled in the art. A nucleic acid analysis for the presence or absence of a genetic marker can be used for the selection of plants or seeds in a breeding population. The analysis may be used to select for genes, QTL, alleles, or genomic regions (haplotypes) that comprise or are linked to a genetic marker. Analysis methods are known in the art and include, but are not limited to, PCR-based detection methods (for example, TAQMAN assays), microarray methods, and nucleic acid sequencing methods. The genes, alleles, QTL, or haplotypes to be selected for can be identified using well known techniques of molecular biology (e.g. Sambrook et al., 1989) and with modifications of classical breeding strategies, for instance as described by Narasimhamoorthy et al. (2007). If the nucleic acids from the plant are positive for a desired genetic marker, the plant can be selfed to create a true breeding line with the same genotype, or it can be crossed with a plant with the same marker or with other desired characteristics to create a sexually crossed hybrid generation. Methods of marker-assisted selection (MAS) using a variety of genetic markers are known in the art.

Marker-assisted introgression involves the transfer of a chromosome region defined by one or more markers from one germplasm to a second germplasm. The initial step in that process is the localization of the genomic region or transgene by gene mapping, which is the process of determining the position of a gene or genomic region relative to other genes and genetic markers through linkage analysis. The basic principle for linkage mapping is that the closer together two genes are on a chromosome, then the more likely they are to be inherited together. Briefly, a cross is generally made between two genetically compatible but divergent parents relative to traits under study. Genetic markers can then be used to follow the segregation of traits under study in the progeny from the cross, often a backcross (BC₁), F₂, or recombinant inbred population. Breeding procedures may be modified as is known in the art in view of the plant species being bred, and its reproductive habits (e.g. selfing or outcrossing).

### B. Plant Breeding

The selection of a suitable recurrent parent is an important step for a successful backcrossing procedure. The goal of a backcross protocol is to alter or substitute a trait or characteristic in the original inbred. To accomplish this, one or more loci of the recurrent inbred parent is modified or substituted with the desired gene from the nonrecurrent (donor) parent, while retaining essentially all of the rest of the desired genetic, and therefore the desired physiological and morphological, constitution of an original inbred. The choice of a particular donor parent will depend on the purpose of the backcross. The exact breeding protocol will depend on the characteristic or trait being altered to determine an appropriate testing protocol. It may be necessary to introduce a test of the progeny to determine if the desired characteristic has been successfully transferred. In the case of plants being bred through the use of molecular markers of the present invention, one may test the progeny lines generated during the backcrossing program as well as using the marker system described herein to select lines based upon markers rather than visual traits, the markers are indicative of a genomic region comprising a favorable haplotype. Nucleic acids extracted from plants are analyzed for the presence or absence of a suitable genetic polymorphism. A non-limiting list of traits of interest for introgression by classical and/or marker-assisted breeding may include tolerance to abiotic stress, tolerance to biotic stress, increased yield, increased nodulation, altered oil content, altered protein content, altered flavonoid content, altered isoflavonoid content, altered maturity group, altered time of flowering, and increased tolerance to wounding, salt, aluminum, cold, heat, drought, oxidative stress, pest infestation, or pathogen infection, among others.

In still another aspect, the invention provides a computer readable data storage medium encoded with computer readable data comprising: one or more nucleotide sequences comprising all or part of a plant transcription factor gene from a plant species, genus, family, tribe, or clade, identified by the above described method wherein the molecular marker is genetically linked to a plant transcriptional factor-encoding gene, or comprises a sequence within a coding or non-coding region of a plant transcriptional factor-encoding gene.

One of ordinary skill in the art will recognize that a variety of techniques may be used to isolate gene segments that correspond to genes previously isolated from other species.

### EXAMPLES

### Example 1

### Plant Material

Seeds from parents of legume mapping populations including M. truncatula, L. japonicus, G. max, L. albus, P. sativum, and P. vulgaris, were planted in the greenhouse (Table 3).

TABLE 3
Entries from multiple legume species evaluated in this study.

| Species | Common name | Number of entries |
|---|---|---|
| Medicago truncatula | Barrel Medics | 8 |
| Medicago sativa | Alfalfa | 8 |
| Glycine max | Soybean | 4 |
| Lotus japonicus | Lotus | 2 |
| Trifolium repens | White Clover | 2 |
| Trifolium pratense | Red Clover | 2 |
| Lupinus albus | Lupin | 2 |
| Vigna radiata | Mung Bean | 2 |
| Pisum sativum | Pea | 2 |
| Phaseolus vulgaris | Common Bean | 2 |

Parents of alfalfa populations segregating for drought (Sledge and Jiang, 2005) and aluminum tolerance, and white clover (Zhang et al., 2007) mapping populations were propagated using cuttings. Young leaf tissue samples were collected, freeze dried, and DNA extracted and purified using the Plant DNeasy kit (Qiagen, Valencia, Calif.). Leaf samples from T. pratense were obtained from Heathcliffe Riday at USDA-ARS in Madison, Wis.

### Example 2

### Primers and PCR Reactions

Two different but complementary approaches are used for primer design. In the first approach, a total of 1084 primer pairs were previously designed and validated to amplify M. truncatula transcription factor sequences (Kakar et al 2008). Medicago TF's were identified by screening 40,000 proteins of IMGAG (International Medicago Genome Annotation Group) release 1 for known or presumed DNA-binding domains using InterPro (www.ebi.ac.uk/interpro). Genomic sequences with DNA-binding domains were used to query NCBI's non-redundant DNA database (www.ncbi.nlm.nih.gov/blast) and the curated protein database UniProt (www.uniprot.org) rather than ESTs for TF gene discovery because those protein sequences are more complete and the set of IMGAG proteins essentially contains no redundancy. The process for developing molecular markers included PCR primer design and testing for gene specificity and amplification efficiency. The M. truncatula genome sequence from IMGAG release 3 (www.medicago.org) may also be utilized to identify approximately 1000 additional Medicago TF's from IMGAG annotated proteins.

The second approach being used develops additional primers from specific transcription factors that result in limited cross-species amplification with the existing primers in the first iteration, which will be used as query sequences. The Database of Arabidopsis Transcription Factors (DATF) (Guo et al., 2005) may be used as a reference. M. truncatula genome sequences and IMGAG predictions will be obtained and analyzed (e.g. from www.medicago.org). Sequences from the preliminary soybean genome sequencing project (Soybean Genome Project; www.phytozome.net/soybean), published soybean protein sequences deposited in NCBI (˜3600 proteins as of October 2008), and unigenes from the Soybean Gene Index (www.compbio.dfci.harvard.edu/tgi/cgi-bin/tgi/gimain.pl?gudb=soybean), e.g. release 13.0 of Jul. 11, 2008, or later, will be translated into peptide sequences. These sequences may later be mapped on the soybean genome. Gene models and corresponding protein sequences in L. japonicus may also be used (www.kazusa.or.jp/lotus). For other legume species without a corresponding genome sequence available, the corresponding Gene Index unigene or ESTs available from NCBI for downstream analysis may be used when available. Whole genome scans may be used to identify putative orthologous genes between legume species based on phylogenetic analysis, gene location, and information on neighboring genes in the genome sequence as previously described (Fulton et al., 2002). The strategy identifies regions of high sequence conservation based on the alignment of multiple legume species and low conservation in the target amplification sequence to increase the likelihood of detecting polymorphism. A 50 by sliding window may be used in the primer design process to identify useful primer sequences. To ensure maximum specificity and efficiency during PCR amplification, criteria used for primer design may include a predicted melting temperature of 58° C. to 61° C., limited self-complementarity and poly-X, and PCR amplicon lengths of 100 to 250 bp. Primers will be evaluated for gene-specificity and amplification efficiency as previously described (Kakar et al., 2008).

PCR amplicons of a total of 1084 transcription-factor based markers (Kakar et al., 2008) obtained using a pooled DNA sample of four alfalfa mapping population parents were separated using agarose gels, stained with ethidium bromide, and visualized using a UV transiluminator. Primers with successful amplification in alfalfa were re-synthesized with an additional 18 nucleotides from the M13 universal primer appended to the 5′ end of the forward primer (Schuelke, 2000) by Integrated DNA Technologies, Inc. (Coralville, Iowa). Equal DNA concentrations for all legume species (20 ng) were used to set up PCR reactions in a total volume of 10 μl and were performed using procedures previously described (Zhang et al., 2008). PCR products were analyzed using the ABI PRISM 3730 Genetic Analyzer with the GeneScan 500 LIZ internal size standard (Applied Biosystems, Foster City, Calif.). PCR amplicons were visualized and analyzed with GeneMapper 3.7 software (Applied Biosystems, Calif., USA) to determine successful amplification and size differences among and within legume species.

### Example 3

### SNP Discovery

PCR reactions producing simple amplification products will be sequenced using the BigDye® terminator v3.1 cycle sequencing kit and an ABI3730 genetic analyzer to confirm amplification of the target sequence and to identify potential SNPs among and within legume species. DNA sequence alignments may be produced with Sequencher™ 4.8, or similar, to survey the parental amplicons for polymorphic sites. PolyBayes, a program primarily designed as a tool for SNP discovery through the analysis of base-wise multiple alignments of clustered DNA sequences (Marth et al., 1999), and methods previously described (e.g. Altshuler et al., 2000) may be used for SNP discovery.

### Example 4

### Molecular Mapping of Markers and Genetic Map Construction

Polymorphic markers in alfalfa, soybean and white clover, including tetraploid lines, can be readily mapped in available mapping populations segregating for multiple traits. The existing SSR linkage maps in these species may be used as a framework for mapping the molecular markers developed from transcription factor sequences. Integrated linkage maps can be constructed using the Kosambi mapping function. The soybean genome sequence (www.phytozome.net/soybean) may be used to integrate the genetic and physical maps in this species. Genetic maps of other plant species are known in the art and may be used similarly.

Genomic DNA from individual genotypes from mapping populations, such as tetraploid alfalfa lines, is obtained as known in the art, for instance using the DNeasy Plant Kit® (QIAGEN, Valencia, Calif., USA). As available, SSR or other polymorphic markers may be used for genotyping the mapping populations as previously described (Narasimhamoorthy et al., 2007). Polymorphic PCR amplification products from SSR and candidate gene-based markers are visualized and scored, for instance using GeneMapper 3.7 software (Applied BioSystems, Carlsbad, Calif). Markers are scored based on segregation ratio in the population to achieve maximum resolution on the parental linkage map. Linkage maps for parent lines are constructed and QTL analysis is performed using phenotypic data to determine the effect and consistency of each QTL detected. Interval mapping for autotetraploid species may be as described by Hackett et al. (2001) and implemented in TetraploidMap (Hackett and Luo, 2003). Multiple regression analysis for each QTL is performed to determine the allele effect at each QTL detected.

### Example 5

### Identification of TF-Associated Primers

Among the first set of 96 primer pairs tested (SEQ ID NOs. 1-192), 88 (92%) primer pairs produced PCR amplification products (Table 4). A total of 711 alleles were identified among all species, with an average of 8 alleles per marker. The PCR amplification product was either the same length in all legume species or the size varied among the legume species in the panel based on the GeneMapper output (FIG. 2). The marker TF56E02 produced a PCR product of the same length in all legume species evaluated, while the size of the amplification product of marker TF56C11 differed among species (FIGS. 2A & B, respectively). From the total number of markers tested so far, the percent of markers with amplification and producing single amplicons was 94%, 52%, 47% and 42% in alfalfa, white clover, L. japonicus and soybean, respectively. An extrapolation of the preliminary results to the total number of primers currently available (Table 4), indicates the potential to contribute an additional 1059, 652, 567, 455, 492, TF-based molecular markers in alfalfa, pea, white clover, soybean, and red clover, respectively. In general, the likelihood of successful amplification decreased with increased phylogenetic distance among species.

TABLE 4
PCR amplification products from multiple legume species evaluated
using 88 primer pairs developed from transcription factor
sequences that yielded amplification products.

| | | Primers with | Polymorphic |
|---|---|---|---|
| | | single | primers |
| Species name | Common name | PCR amplicon | (size only) |
| M. truncatula | Barrel medic | 86 | 25 |
| M. sativa | Alfalfa | 83 | 22 |
| P. sativum | Pea | 53 | 10 |
| T. repens | White clover | 46 | 5 |
| L. japonicus | Lotus | 41 | 2 |
| L. albus | Lupin | 41 | 6 |
| T. pratense | Red clover | 40 | 7 |
| P. vulgaris | Common bean | 39 | 13 |
| G. max | Soybean | 37 | 23 |
| V. radiata | Mung bean | 30 | 21 |
| A. thaliana | Arabidopsis | 42 | 10 |

All of the compositions and methods disclosed and claimed herein can be made and executed without undue experimentation in light of the present disclosure. While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### References

The following references are incorporated herein by reference:
Altshuler et al., Nature 407:513-516, 2000.
Ban et al., Pl. Cell Physiol. 48:958-970, 2007.
Benefito et al., Plant J. 55:504-513, 2008.
Borevitz et al., Gen. Res. 13:513-523, 2003.
Cai et al., Pl. Physiol. 145:98-105, 2007.
Dai et al., Pl. Physiol. 143:1739-1751, 2007.
Deluc et al., Pl. Physiol. 147:2041-2053, 2008.
Fulton et al., Pl. Cell 14:1457-1467, 2002.
Guo et al., Bioinformatics 21:2568-2569, 2005.
Hackett, and Luo, J. Heredity 94:358-359, 2003.
Hackett et al., Genetics 159:1819-32, 2001.
Huynen and Bork, Proc Natl Acad Sci USA 95:5849-5856, 1998.
Huynen et al., Genome Research, 10:1204-1210, 2000.
Iuchi et al., Proc. Nat. Acad. Sci. USA 104:9900-9905, 2007.
Kakar et al. Plant Methods 4:18, 2008.
Li et al., Pl. Cell 20:2238-2251, 2008.
Libault et al., Mol. Pl.-Microbe Interact. 20:900-911, 2007.
Marth et al., Nat. Genet. 23:452-456, 1999.
Morley et al., Nature 430:743-747, 2004.
Mueller et al., Plant Cell 20:768-785, 2008.
Narasimhamoorthy et al., TAG 114:901-913, 2007.
Paterson et al., Nature 335:721-726, 1988.
Peakall et al., Mol. Biol. Evol. 15:1275-1287, 1998.
Raffaele et al., Pl. Cell 20:752-767, 2008.
Reichmann et al., Science 290:2105-2110, 2000.
Sambrook et al., (ed.), Molecular Cloning, Cold Spring Harbor Laboratory Press, Cold Spring Harbor, N.Y., 1989.
Schauser et al., Nature 402:191-195, 1999.
Schuelke, Nat. Biotechnol. 18:233-234, 2000.
Sledge and Jiang, TAG 111:980-992, 2005.
Thompson et al., Nucleic Acids Res. 22:4673-4680, 1994.
Udvardi et al., Pl. Physiol. 144:538-549, 2007.
West et al., Genetics 175:1441-1450, 2007.
Zhang et al., Plant J. 42:689-707, 2005.
Zhang et al., TAG 114:1367-1378, 2007.
Zhang et al., Plant Methods 4:19, 2008.
Zhu et al., Pl. Physiol. 137:1189-1196, 2005.

What is claimed is:

1. A method for detecting the location of a locus of interest in a plant comprising:
(a) identifying a sequence from a first plant transcription factor gene of a plant of a first plant species, wherein the transcription factor gene is genetically linked to a locus of interest in said plant;
(b) detecting the presence of a sequence from an orthologous plant transcription factor gene in a plant of a second plant species; wherein the orthologous plant transcription factor gene is genetically linked to an orthologous locus of interest in the plant of the second plant species, whereby the presence of the orthologous plant transcription factor gene is indicative of the presence of the orthologous locus of interest in the plant.

2. The method of claim 1, wherein identifying a sequence from a first plant transcription factor gene and/or detecting the presence of a sequence from an orthologous plant transcription factor gene comprises detecting the presence of a polymorphism in said first plant transcription factor gene and/or said orthologous plant transcription factor gene.

3. The method of claim 1, wherein the first and second plant species are legume (Leguminosae) species or grass species.

4. The method of claim 3, wherein the first and second plant species are Galegoid legume species.

5. The method of claim 3, wherein the first and second plant species are Phaseoloid legume species.

6. The method of claim 3, wherein the first plant species is a Phaseoloid legume species and the second plant species is a Galegoid legume species.

7. The method of claim 3, wherein the first plant species is a Galegoid legume species and the second plant species is a Phaseoloid legume species.

8. The method of claim 3, wherein the first and second plant species are selected from members of the group consisting of the tribes Viceae, Trifoleae, Cicereae, Loteae, and Phaseoleae.

9. The method of claim 3, wherein the first and second plant species are selected from the members of the group consisting of the genera Lens, Vicia, Pisum, Melilotus, Trifolium, Medicago, Cicer, Lotus, Phaseolus, Vigna, Glycine, Arachis, and Cajanus.

10. The method of claim 3, wherein the first and second plant species are selected from members of the group consisting of the genera Medicago, Lotus, Phaseolus, Glycine, Festuca, Panicum, and Triticum.

11. The method of claim 3, wherein the first and second plant species are Medicago sp. or Glycine sp.

12. An isolated nucleic acid molecule comprising a sequence selected from the group consisting of: SEQ ID NOs:1-192.

13. The method of claim 1, wherein detecting the presence of a plant transcription factor gene or an orthologous plant transcription factor gene comprises a technique selected from the group consisting of: PCR, nucleotide hybridization, single strand conformational polymorphism analysis, denaturing gradient gel electrophoresis, cleavage fragment length polymorphism analysis and/or DNA sequencing.

14. The method of claim 13, wherein detecting the presence of a plant transcription factor gene in a first plant species and detecting the presence of an orthologous plant transcription factor gene in a second plant species comprises utilizing the same technique for each species.

15. The method of claim 14, wherein the technique comprises utilization of a primer pair or a hybridization probe.

16. The method of claim 15, wherein the primer pair or hybridization probe utilized for each plant species comprises the same nucleotide sequence.

17. A method for breeding a plant comprising: introgressing a trait genetically linked to the first or second locus identified according to the method of claim 1 into the genome of a plant by performing marker-assisted selection.

18. The method of claim 17, wherein marker-assisted selection comprises PCR, nucleotide hybridization, single strand conformational polymorphism analysis, denaturing gradient gel electrophoresis, cleavage fragment length polymorphism analysis and/or DNA sequencing.

19. The method of claim 17, wherein the trait is selected from the group consisting of: tolerance to abiotic stress, tolerance to biotic stress, increased yield, increased nodulation, altered oil content, altered protein content, altered flavonoid content, maturity group, and time of flowering.

20. The method of claim 19, wherein the trait confers increased tolerance to wounding, salt, cold, heat, drought, oxidative stress, aluminum, pest infestation, or pathogen infection.

21. A computer readable data storage medium encoded with computer readable data comprising: one or more nucleotide sequences identified according to the method of claim 1.
